SEQUENCE LISTING (1 sequence)

SEQ ID NO: 1 - protein, 147 aa, Homo sapiens
MVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

(19) United States
(12) Patent Application Publication
(10) Pub. No.: US 2025/0339497 A1
(43) Pub. Date: Nov. 6, 2025

(54) METHODS FOR TREATING MOOD DISORDERS BY ADMINISTERING A LEPTIN RECEPTOR AGONIST

(71) Applicant: Universität Duisburg-Essen, Essen (DE)

(72) Inventors: Johannes HEBEBRAND, Essen (DE); Jochen ANTEL, Essen (DE); Gertraud GRADL-DIETSCH, Essen (DE)

(21) Appl. No.: 18/854,864

(22) PCT Filed: Apr. 6, 2023
(86) PCT No.: PCT/EP2023/059245

§ 371 Date: Oct. 7, 2024

(30) Foreign Application Priority Data
Apr. 7, 2022 (LU) LU501817

(51) Int. Cl. A61K 38/22; A61K 31/135; A61K 31/335; A61K 31/497; A61K 31/55; A61K 31/553; A61P 25/24

(52) U.S. Cl. CPC A61K 38/2264; A61K 31/135; A61K 31/335; A61K 31/497; A61K 31/55; A61K 31/553; A61P 25/24

(57) ABSTRACT

The invention concerns methods for treating mood disorders such as depressive disorder or episodes, or other mood disorders with current depressive episodes, by administration of a leptin receptor agonist, such as metreleptin. The invention further shows amelioration of depressive disorder or episode and related conditions in patients suffering from depression as a primary disorder, or a depressive disorder or episode as a secondary disorder and/or comorbidity. The invention also concerns methods for treating a depressive episode, or a depressive disorder.

### FIELD OF THE INVENTION

The invention concerns methods for treating mood disorders such as depressive disorder or episodes, or other mood disorders with current depressive episodes, by administration of a leptin receptor agonist, such as metreleptin. The invention further shows amelioration of depressive disorder or episode and related conditions in patients suffering from depression as a primary disorder, or a depressive disorder or episode as a secondary disorder and/or comorbidity.

### DESCRIPTION

Human studies have yielded conflicting results for an association of leptin levels with depression with some authors reporting lower leptin levels in depressive patients compared to healthy controls, others describing elevated or normal levels (Ge und Fan et al. 2018). Associations between depression and low as well as high serum leptin levels may apply (Lawson et al. 2012, Cao et al. 2018); corresponding clinical entities include anorexia nervosa (AN) and hypothalamic amenorrhea and on the opposite side of the body mass index (BMI) distribution obesity. Off-label treatment with recombinant human leptin (metreleptin) led to a pronounced reduction of depressive symptoms in single patients with AN with comorbid depression (Antel J, et al., Eur Child Adolesc Psychiatry. 2021 May 9:1-7. doi: 10.1007/s00787-021-01778-7. Epub ahead of print. PMID: 33966118; Milos G, et al Transl Psychiatry. 2020 Aug. 27; 10(1):303. doi: 10.1038/s41398-020-00977-1. PMID: 32855384; PMCID: PMC7453199; Hebebrand J et al Z Kinder Jugendpsychiatr Psychother. 2021 January;49(1):1-5. German. doi: 10.1024/1422-4917/a000775. PMID: 33423560.).

Leptin is a polypeptide hormone playing a critical role in regulating body weight, food intake, and energy metabolism (Ge et al., 2018; Otero et al., 2006). It is produced and secreted by white adipose tissue (Lu, 2007; Otero et al., 2006), with circulating levels being increased in proportion to body fat stores (Fried et al., 2000) but also influenced by sex (Chan et al., 2002).

Leptin crosses the blood brain barrier and binds to a specific leptin receptor (LepRb) that is distributed in several brain regions (hypothalamic and thalamic regions, but also in other brain regions such as the hippocampus, substantia nigra pars compacta, cerebellum and in some cortical areas) (Ge et al., 2018; Tartaglia et al., 1995; Zou et al., 2019). This brain distribution of LepRb suggests that leptin, besides regulating feeding behaviour and energy expenditure, may be involved in other neural functions and the reward system in particular (Ge et al., 2018).

Lipodystrophy (LD) syndromes are a group of rare heterogeneous disorders classified as an orphan disease and characterized by a reduction in subcutaneous fat (Quinn et al., 2021) and a selective deficiency in functional adipose tissue in the absence of nutritional deprivation or catabolic state which causes reduced energy storage capacity and a deficiency of adipokines such as leptin (Araújo-Vilar & Santini, 2019; Brown et al., 2016; Cook et al., 2021; Melvin et al., 2019)

Treatment with recombinant human leptin (r-metHuLeptin; metreleptin) of patients with chronic leptin deficiency (CLD) entails an increased satiety and satiation within seven days after its initiation; weight loss is pronounced over time (Farooqi et al., 1999; Licinio et al., 2004; Roth, von Schnurbein, Elfers, Moss, & Wabitsch, 2018). A reduced irritability around eating was noted (Roth et al., 2018). A nine year old female no longer secretly sought food or demanded food between meals (Farooqi et al., 1999). We are aware of two studies on individuals with CLD that report on potential psychological effects of metreleptin treatment that extend beyond effects on satiety and satiation: i) in three adults with CLD, “non-ingestive behavior changed dramatically within two weeks” of metreleptin treatment and prior to substantial weight loss (Licinio et al., 2004). These adults did not have elevated depression or anxiety scores prior to treatment; the authors thus precluded changes in mood or anxiety as an explanation for the observed mental effects, which they describe as a change in behavior and interpersonal attitudes from a baseline of infantile and docile to assertive and adult-like. ii) After metreleptin treatment of a five year old boy for a two year period, substantial increments in the rates of development in neurocognitive domains were observed.

Hence, there is still a need for therapeutic interventions to treat or ameliorate depression and related disorders.

### BRIEF DESCRIPTION OF THE INVENTION

Generally, and by way of brief description, the main aspects of the present invention can be described as follows:

In a first aspect, the invention pertains to a method for treating a subject suffering from a mood disorder, comprising an administration of a therapeutically effective amount of leptin, or a leptin- and/or leptin receptor agonist, or a leptin analog or derivative thereof, to the subject and thereby ameliorating the mood disorder.

In a second aspect, the invention pertains to a use of a compound or composition in the treatment of a mood disorder, wherein the compound or composition is comprising a leptin, or a leptin- and/or leptin receptor agonist, or a leptin analog or derivative thereof.

In a third aspect, the invention pertains to a method for treatment of a mood disorder in a subject, the method comprising administering a therapeutically effective amount of leptin, or a leptin- and/or leptin receptor agonist, or a leptin analog or derivative thereof, to the subject, together with an administration of at least one additional antidepressant.

### DETAILED DESCRIPTION OF THE INVENTION

In the following, the elements of the invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

The present invention provides compounds and compositions for use, as well as uses and methods of treatment, of mood disorders such as a depressive disorder, or other mood disorder with a preferably current depressive episode, by administering leptin receptor agonists. The described invention constitutes a surprising teaching since while the association of endogenous leptin and depression in the prior art is very inconsistent, under discussion and at best only fragmentarily understood. The invention provides for the first-time proof and technical teaching that independent of the involvement of endogenous leptin levels, mood disorders, preferably such with depressive episodes, can be targeted by the administration of leptin agonists, such as metreleptin.

In a first aspect, the invention pertains to a method for treating a subject suffering from a mood disorder, comprising an administration of a therapeutically effective amount of leptin, or a leptin- and/or leptin receptor agonist, or a leptin analog or derivative thereof, to the subject and thereby ameliorating the mood disorder.

In a second aspect, the invention pertains to a use of a compound or composition in the treatment of a mood disorder, wherein the compound or composition is comprising a leptin, or a leptin- and/or leptin receptor agonist, or a leptin analog or derivative thereof.

The invention surprisingly provides a treatment with a leptin-analog as therapeutically effective for mood disorders or depressive disorders or depressive episodes in connection with a mood disorder. So far data on leptin in humans are heterogenous and contradictory and do not suggest that leptin analogues including metreleptin may have an antidepressant effect. In particular, several studies have investigated serum leptin levels in patients with major depressive disorder. Both high and low leptin levels have been associated with this disorder. In a first meta-analysis (Carvalho, A. F., Rocha, D. Q., McIntyre, R. S., Mesquita, L. M., Kohler, C. A., Hyphantis, T. N., Berk, M. (2014). Adipokines as emerging depression biomarkers: a systematic review and meta-analysis. J Psychiatr) of studies comparing leptin levels in patients and healthy controls both body mass index (BMI) and difference in BMI between patients and controls explained heterogeneity of leptin. Leptin levels were significantly higher in individuals with mild/moderate depression versus controls. In addition, leptin serum levels did not change after antidepressant treatment. However, the low sample size precluded a meta-regression analysis for this particular analysis. In the second and more recent meta-analysis a total, of 4,372 (52.3%) subjects with MDD and 3,984 (47.7%) healthy controls were compared (Cao, B., Chen, Y., Brietzke, E., Cha, D., Shaukat, A., Pan, Z., . . . McIntyre, R. S. (2018). Leptin and adiponectin levels in major depressive disorder: A systematic review and meta-analysis. J Affect Disord, 238, 101-110. doi:10.1016/j.jad.2018.05.008). The two groups did not differ in leptin levels. Post-hoc analyses suggested that males with lower leptin levels may have an increased likelihood of developing MDD. Overall, the development of this invention is therefore, surprising.

The essential features of the invention lie in the treatment of a depressive episode for example in connection with a mood disorder such as “depression”. Such an episode is a period of at least 2 weeks during which there is either a depressed mood or the loss of interest or pleasure in nearly all activities. The individual must also experience at least four additional symptoms drawn from a list that includes changes in appetite or weight, sleep, and psychomotor activity; decreased energy; feelings of worthlessness or guilt; difficulty thinking, concentrating, or making decisions; or recurrent thoughts of death or suicidal ideation, plans or attempts. To be considered a major depressive episode, a symptom must have clearly worsened compared with the person's pre-episode status. The symptoms must persist for most of the day, nearly every day, for at least 2 consecutive weeks. The episode must be accompanied by clinically significant distress or impairment in social, occupational or other important areas of functioning (Diagnostic and Statistical Manual of Mental Disorders 4th Ed. DSM-IV, Pub. American Psychiatric Association, Washington, D.C.; p. 320, 327, 344-345). A “major depressive disorder” generally refers to a single or recurrent Major Depressive Episode which is not better accounted for by Schizophrenia, Delusional Disorder, or Psychotic Disorder Not Otherwise Specified, and also there has never been a Manic Episode, a Mixed Episode or a Hypomanic Episode (Diagnostic and Statistical Manual of Mental Disorders, fifth edition. DSM-V, American Psychiatric Association, 2013. DSM-5; Diagnostic and statistical manual of mental disorders, 5 ed, Washington, DC). Tables 1, 2 and 3 further define the criteria for Major Depressive Episodes/Disorders: Criteria for Major Depressive Episode (Table 1); a Single Episode of a Major Depressive Disorder (Table 2) and recurrent Major Depressive Disorder (Table 3). The diagnosis is generally based on evaluation by a qualified physician, generally a psychiatrist or by a psychologist. A “minor depressive disorder”, also referred to as “dysthymia”, has the characteristics of a major depressive disorder but presents itself without the intensity or severity of the symptoms associated with a “major depressive disorder”. “Late Life Major Depression”, referred to as “LLMD” or “late-onset depression” refers to depression, for example, the major and minor depressive disorders and depressive episodes described above, that occurs in a subject at about 60 years of age or older. The “risk factors” for depression include female gender, unmarried status, having stressful life events and lack of a social support network. Major depressive disorder is characterized by any of a number of symptoms, including persistent sadness or anxiety, or feelings of emptiness, hopelessness, pessimism, guilt, worthlessness, or helplessness.

As used herein, “treatment” is an approach for obtaining beneficial or desired clinical results. For purposes of this invention, beneficial or desired clinical results include, but are not limited to, one or more of the following: improving, lessening severity, alleviation of one or more symptoms associated with a disease, preferably in context of the invention the disease is a mood disorder, for example a depressive disorder selected from the group consisting of bipolar disorders, dysthymia, cyclothymia, and premenstrual syndrome. The depressive episode can be mild, moderate or severe.

In context of the present invention a mood disorder is selected from a disorder categorised as mood disorder according to the ICD 11 classification of the World Health Organisation (see also “https://icd.who.int/en” in its version of Mar. 17 2022). Mood disorders are defined therein as a superordinate grouping of Bipolar and Depressive Disorders. Mood disorders are defined according to particular types of mood episodes and their pattern over time. The primary types of mood episodes are depressive episode, manic episode, mixed episode, and hypomanic episode. Mood episodes are not independently diagnosable entities, and therefore do not have their own diagnostic codes. Rather, mood episodes make up the primary components of most of the Depressive and Bipolar Disorders. In context of the present invention such individuals or subjects shall be treated that suffer such a mood disorder and are characterised by a current depressive episode—the invention surprisingly offers amelioration of the depressive episode. In particular, a patient suffering from a current maniac or hypomanic episode shall be excluded from group of subjects or individuals receiving a treatment of the herein described invention. In other words, a subject shall not have a current maniac episode.

In a preferred embodiment, the treatment is a treatment of the depressive disorder in the subject using the compound or composition of the invention. In another embodiment the subject may be a subject suffering from bulimia nervosa as comorbidity with a depressive episode or depressive disorder.

In particular, a patient suffering from a depressive episode as a comorbidity may be treated for the depressive episode by the present invention. Such patients often suffer from one-to-many other disorders. Such patients who are diagnosed as having a mood disorder with depressive episode which is treatable according to the invention may be patients who primarily suffer from mental disorders with the subordinate diagnostic category (ICD-11; 06) being, anxiety or fear related disorders, disorders specifically associated with stress, neurodevelopmental disorders, schizophrenia or other primary psychotic disorders, catatonia, obsessive compulsive or related disorders, disorders of bodily distress or bodily experience, dissociative disorders, disorders due to substance use or addictive behaviours, impulse control disorders, disruptive behaviour or dissocial disorders, personality disorders and related traits, mental or behavioural disorders associated with pregnancy, childbirth or the puerperium, factitious disorders, paraphilic disorders, psychological or behavioural factors affecting disorders or diseases classified elsewhere, secondary mental or behavioural syndromes associated with disorders or diseases classified elsewhere.

Other diagnostic subordinates include but are not limited to sleep-wake disorders (ICD-11; 07), diseases of the nervous system (ICD-11; 08), diseases of the digestive system (ICD-11; 13).

The depressive disorder may also be associated with any one of the “Symptoms, signs or clinical findings, not elsewhere classified” (ICD-11; 21) including for instance cachexia (ICD-11; MG20) or fatigue (MG22).

In some embodiments, the subject may suffer in addition from lipodystrophy, however, the treatment is administered for therapy of the depressive episode. The term “lipodystrophy” refers to any conditions characterised by a disturbance of lipid (fat) metabolism that involves the partial or total absence of fat and the abnormal deposition and distribution of fat in the body. The term also includes the more specific term “lipoatrophy” which is used when describing the loss of fat from one area (e.g. the face). Lipodystropies can be a possible side effect of HIV medication (mainly the use of protease inhibitors). Other lipodystropies manifest as the excess or lack of fat in various regions of the body. These include but are not limited to having sunken cheeks, “humps” on the back or back of the neck and small lumps or dents in the skin formed by repetitive injections in the same spot (e.g. insulin use in diabetics). Lipodystrophy can be genetic or acquired, for example LD can also be caused by metabolic abnormalities due to genetic issues. These are often characterised by insulin resistance. Compounds according to the invention for the treatment of depressive disorder and lipodystrophy may be co-administered with other moieties that are used for such treatment, including, for example, poly-L-lactic acid (e.g. Sculptra).

In some preferred embodiments, the lipodystrophy syndrome in the subject involves distribution of fat loss, which can be generalized (GLD) or partial (PLD.

“Ameliorating” a disease or one or more symptoms of the disease means a lessening or improvement of one or more symptoms associated with the disease as compared to not administering a leptin receptor agonist. “Ameliorating” also includes shortening or reduction in duration of a symptom.

As used herein, an “effective dosage” or “effective amount” of drug, compound, or pharmaceutical composition is an amount sufficient to effect beneficial or desired results. For prophylactic use, beneficial or desired results include results such as eliminating or reducing the risk, lessening the severity, or delaying the outset of the disease, including biochemical, histological and/or behavioural symptoms of the disease, its complications and intermediate pathological phenotypes presenting during development of the disease, in particular as disclosed herein elsewhere. For therapeutic use, beneficial or desired results include clinical results such as reducing intensity, duration, or frequency of attack of the disease, and decreasing one or more symptoms resulting from the disease (biochemical, histological and/or behavioural), including its complications and intermediate pathological phenotypes presenting during development of the disease, increasing the quality of life of those suffering from the disease, decreasing the dose of other medications required to treat the disease, enhancing effect of another medication, and/or delaying the progression of the disease of patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

The term “leptin receptor agonist” refers to a leptin protein, a fragment of a leptin protein having physiological properties of the leptin protein, analog (or variant) leptin protein or variant of a fragment of a leptin protein fragment, having physiological properties of the leptin protein, a leptin receptor agonist being a leptin mimic or any combinations thereof.

Such compounds may be the full (human) leptin protein having a sequence as shown in Zhang Y, Proenca R, Maffei M, Barone M, Leopold L, Friedman J M. Positional cloning of the mouse obese gene and its human homologue. Nature 1994 1:372 (6505):425-432. and as indicated in the UniProt database under accession number P41159 in the version of Aug. 24, 2021. In other embodiments the leptin is a pegylated (PEG)-leptin.

Preferably, the leptin analog is metreleptin, which is also a preferred leptin receptor agonist according to the invention.

Metreleptin is an active substance from the group of leptin analogues for the treatment of complications of leptin deficiency in patients with congenital or acquired generalized lipodystrophy. It is a derivative of the polypeptide hormone leptin, which is produced by adipose tissue, inhibits appetite, and decreases insulin resistance. The drug is administered as a subcutaneous injection. Metreleptin is a recombinant human leptin analog derived from E. coli by biotechnological methods. It differs from natural (human) leptin in having an additional methionine at the N-terminus. Metreleptin is an unglycosylated polypeptide of 147 amino acids with a disulfide bridge between Cys-97 and Cys-147 and a molecular weight of approximately 16.15 kDa. Its sequence is shown in SEQ ID NO: 1.

The compounds of the invention can be administered as a “therapeutic composition”, which can refer to any compounds administered to treat or prevent a disease or a symptom(s) thereof, such as complications associated with weight loss or underweight. For example, aspects of the invention are drawn towards uses of therapeutic compositions comprising leptin and/or leptin receptor agonists, such as metreleptin.

In embodiments, the therapeutic composition can comprise human recombinant leptin and derivatives or fragments thereof. For example, Metreleptin (Myalept®) is an FDA-approved treatment for generalized and familial dyslipidemia, and thus could readily be repurposed to treat or prevent the loss of the counter-regulatory response in diabetes patients. Thus, embodiments of the invention comprise treatment strategies for utilizing metreleptin to treat or prevent neuropsychiatric conditions associated with weight loss or underweight as disclosed herein.

Compositions as utilized herein can also be provided as therapeutic or prophylactic combination compositions that comprise leptin, fragments thereof, and/or leptin receptor agonists, and one or more additional active agents. For example, a therapeutic or prophylactic combination composition can comprise leptin and an anti-depressant that can be used to prevent and/or treat depressive disorders as defined herein, and/or lipodystrophy.

The therapeutic compositions can be incorporated into pharmaceutical compositions suitable for administration. Such compositions can comprise leptin, fragments thereof, and/or leptin receptor agonists, and a pharmaceutically acceptable carrier. Thus, in some embodiments, the compounds of the invention are present in a pharmaceutical composition. According to the invention, a pharmaceutically acceptable carrier can comprise any and all solvents, dispersion media, coatings, antibacterial and antifungal agents, isotonic and absorption delaying agents, and the like, compatible with pharmaceutical administration. Non-limiting examples of pharmaceutically acceptable carriers comprise solid or liquid fillers, diluents, and encapsulating substances, including but not limited to lactose, dextrose, sucrose, sorbitol, mannitol, xylitol, erythritol, maltitol, starches, gum acacia, alginate, gelatin, calcium phosphate, calcium silicate, cellulose, methyl cellulose, microcrystalline cellulose, polyvinylpyrrolidone, water, methyl benzoate, propyl benzoate, talc, magnesium stearate, and mineral oil. The use of such media and agents for pharmaceutically active substances is well known in the art. Any conventional media or agent that is compatible with the active compound can be used. Supplementary active compounds can also be incorporated into the compositions.

The term “therapeutically effective amount” can refer to those amounts that, when administered to a subject in view of the nature and severity of that subject's disease or condition, will have a desired therapeutic effect, e.g., an amount which will cure, prevent, inhibit, or at least partially arrest or partially prevent a target disease or condition. In some embodiments, the term “therapeutically effective amount” or “effective amount” can refer to an amount of a therapeutic agent that when administered alone or in combination with an additional therapeutic agent to a cell, tissue, or subject is effective to prevent or ameliorate the disease or condition such as depression or related disorder. A therapeutically effective dose further refers to that amount of the therapeutic agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously.

A therapeutically effective dose can depend upon a number of factors known to those of ordinary skill in the art. The dose(s) can vary, for example, depending upon the identity, size, and condition of the subject or sample being treated, further depending upon the route by which the composition is to be administered, if applicable, and the effect which the practitioner desires. These amounts can be readily determined by the skilled artisan.

In some embodiments, the therapeutically effective amount is at least about 0.1 mg/kg body weight, at least about 0.25 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 3500 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. The dosage can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion.

In further embodiments, metreleptin is administered at 0.06 mg/kg to 10 mg per day, once daily, preferably wherein metreleptin is administered at about 3 to 10 mg per day, once daily.

In accordance with some embodiments of the invention the leptin, or the leptin- and/or leptin receptor agonist, or the leptin analog or derivative thereof, is administered at: from about 1 microgram per day, from about 5 micrograms per day, about 10 micrograms per day, from about 50 micrograms per day, or from about 100 micrograms per day; to about 100 micrograms per day, to about 500 micrograms per day, to about 1 milligram per day, to about 5 milligrams per day, to about 50 milligrams per day, or to about 100 milligrams per day; or from about 0.01 milligram per kilogram to about 0.3 milligram, or from about 0.01 milligram per kilogram to about 20 milligrams per kilogram.

In preferred embodiments of the invention the metreleptin is administered at: from about 1 milligram per day, from about 5 milligrams per day, to about a maximum of 10 milligrams per day; or from about 0.06 milligram per kilogram per day to about 2.5 milligrams per kilogram per day.

In certain embodiments of the invention the subject is a human and has a serum leptin level below the 50th percentile, more preferably below the 5th percentile, and most preferably below the 1st percentile, according to the subject's age and sex, and wherein the threshold is according to the following Tables A and B:

TABLE A
Girls (left) or boys (right) at the age of (5.8-19.9 y)

| Percentile/Perzentile (μg/L) | | | | | | Percentile/Perzentile (μg/L) | | | | | |
|---|---|---|---|---|---|---|---|---|---|---|---|
| BMI (kg/m²) | 1 | 5 | 50 | 95 | 99 | BMI (kg/m²) | 1 | 5 | 50 | 95 | 99 |
| 11 | 0.32 | 0.41 | 0.73 | 1.29 | 1.63 | 11 | 0.03 | 0.05 | 0.18 | 0.58 | 0.94 |
| 12 | 0.41 | 0.52 | 0.92 | 1.63 | 2.06 | 12 | 0.04 | 0.07 | 0.22 | 0.71 | 1.16 |
| 13 | 0.52 | 0.66 | 1.16 | 2.07 | 2.61 | 13 | 0.49 | 0.08 | 0.27 | 0.88 | 1.43 |
| 14 | 0.65 | 0.83 | 1.47 | 2.61 | 3.31 | 14 | 0.06 | 0.10 | 0.33 | 1.08 | 1.75 |
| 15 | 0.83 | 1.05 | 1.87 | 3.31 | 4.19 | 15 | 0.07 | 0.12 | 0.40 | 1.32 | 2.16 |
| 16 | 1.05 | 1.33 | 2.36 | 4.19 | 5.30 | 16 | 0.09 | 0.15 | 0.49 | 1.63 | 2.65 |
| 17 | 1.33 | 1.68 | 2.99 | 5.30 | 6.71 | 17 | 0.11 | 0.18 | 0.61 | 2.00 | 3.26 |
| 18 | 1.68 | 2.13 | 3.78 | 6.71 | 8.49 | 18 | 0.14 | 0.23 | 0.75 | 2.46 | 4.01 |
| 19 | 2.13 | 2.69 | 4.79 | 8.5 | 10.8 | 19 | 0.17 | 0.28 | 0.92 | 3.03 | 4.93 |
| 20 | 2.69 | 3.41 | 6.06 | 10.7 | 13.6 | 20 | 0.21 | 0.34 | 1.13 | 3.72 | 8.08 |
| 21 | 3.41 | 4.31 | 7.67 | 13.61 | 17.2 | 21 | 0.26 | 0.42 | 1.39 | 4.58 | 7.46 |
| 22 | 4.32 | 5.46 | 9.71 | 17.2 | 21.8 | 22 | 0.32 | 0.52 | 1.71 | 5.63 | 9.17 |
| 23 | 5.46 | 6.91 | 12.3 | 21.8 | 27.6 | 23 | 0.39 | 0.64 | 2.10 | 6.92 | 11.3 |
| 24 | 6.91 | 8.75 | 15.6 | 27.6 | 34.9 | 24 | 0.48 | 0.78 | 2.58 | 8.51 | 13.9 |
| 25 | 8.75 | 11.1 | 19.7 | 34.9 | 44.2 | 25 | 0.59 | 0.96 | 3.18 | 10.5 | 17.0 |
| 26 | 11.1 | 14.0 | 24.9 | 44.2 | 56.0 | 26 | 0.73 | 1.19 | 3.91 | 12.9 | 21.0 |
| 27 | 14.0 | 17.7 | 31.8 | 58.0 | 70.9 | 27 | 0.89 | 1.46 | 4.80 | 15.8 | 25.8 |
| 28 | 17.8 | 22.5 | 39.9 | 70.9 | 89.7 | 28 | 1.10 | 1.79 | 5.90 | 19.4 | 31.7 |
| 29 | 22.5 | 28.4 | 50.5 | 89.7 | 114.0 | 29 | 1.35 | 2.20 | 7.25 | 23.9 | 39.0 |
| 30 | 28.4 | 36.0 | 63.9 | 114.0 | 144.0 | 30 | 1.66 | 2.71 | 8.93 | 29.4 | 48.0 |
| 31 | 36.0 | 45.6 | 80.9 | 144.0 | | 31 | 2.05 | 3.33 | 11.0 | 36.2 | 58.9 |
| 32 | 45.6 | 57.7 | 80.2 | 144.0 | | 32 | 2.51 | 4.09 | 13.5 | 44.5 | 72.4 |
| 33 | 57.7 | 73.0 | 102.0 | | | 33 | 3.09 | 5.04 | 16.6 | 54.7 | 89.1 |
| 34 | 73.0 | 92.4 | 130.0 | | | 34 | 3.80 | 6.20 | 20.4 | 67.2 | 109.0 |
| 35 | 92.4 | 117.0 | | | | 35 | 4.88 | 7.62 | 25.1 | 82.6 | 134.0 |
| 36 | 117.0 | 148.0 | | | | 36 | 5.75 | 9.37 | 30.9 | 101.0 | |
| 37 | 148.0 | | | | | 37 | 7.07 | 11.5 | 37.9 | 124.0 | |
| | | | | | | 38 | 8.7 | 14.2 | 46.7 | | |
| | | | | | | 39 | 10.7 | 17.4 | 57.4 | | |
| | | | | | | 40 | 13.1 | 21.4 | 70.5 | | |

TABLE B
Adult women (left) or adult men (right)

| Percentile/Perzentile (μg/L) | | | | | | Percentile/Perzentile (μg/L) | | | | | |
|---|---|---|---|---|---|---|---|---|---|---|---|
| BMI (kg/m²) | 1 | 5 | 50 | 95 | 99 | BMI (kg/m²) | 1 | 5 | 50 | 95 | 99 |
| 11 | 0.46 | 0.65 | 1.53 | 3.59 | 5.10 | 11 | 0.03 | 0.05 | 0.15 | 0.44 | 0.69 |
| 12 | 0.53 | 0.75 | 1.77 | 4.16 | 5.80 | 12 | 0.04 | 0.06 | 0.18 | 0.55 | 0.87 |
| 13 | 0.61 | 0.87 | 2.05 | 4.82 | 6.83 | 13 | 0.05 | 0.08 | 0.23 | 0.69 | 1.08 |
| 14 | 0.71 | 1.01 | 2.37 | 5.58 | 7.91 | 14 | 0.06 | 0.09 | 0.28 | 0.85 | 1.34 |
| 15 | 7.82 | 1.17 | 2.75 | 6.46 | 9.17 | 15 | 0.07 | 0.12 | 0.35 | 1.06 | 1.67 |
| 16 | 0.95 | 1.35 | 3.18 | 7.48 | 10.61 | 16 | 0.09 | 0.15 | 0.44 | 1.33 | 2.09 |
| 17 | 1.10 | 1.57 | 3.68 | 8.68 | 12.3 | 17 | 0.12 | 0.18 | 0.55 | 1.65 | 2.60 |
| 18 | 1.28 | 1.81 | 4.27 | 10.0 | 14.2 | 18 | 0.14 | 0.23 | 0.68 | 2.06 | 3.24 |
| 19 | 1.48 | 2.10 | 4.94 | 11.6 | 16.5 | 19 | 0.18 | 0.28 | 0.85 | 2.57 | 4.04 |
| 20 | 1.71 | 2.43 | 5.72 | 13.4 | 19.1 | 20 | 0.22 | 0.35 | 1.06 | 3.20 | 5.03 |
| 21 | 1.99 | 2.82 | 6.62 | 15.6 | 22.1 | 21 | 0.23 | 0.44 | 1.32 | 3.98 | 6.27 |
| 22 | 2.30 | 3.28 | 7.67 | 18.0 | 25.8 | 22 | 0.35 | 0.54 | 1.64 | 4.97 | 7.81 |
| 23 | 2.66 | 3.78 | 8.88 | 20.9 | 29.3 | 23 | 0.43 | 0.78 | 2.05 | 6.19 | 9.73 |
| 24 | 3.08 | 4.38 | 10.3 | 24.2 | 34.3 | 24 | 0.54 | 0.85 | 2.55 | 7.71 | 12.1 |
| 25 | 3.57 | 5.07 | 11.9 | 28.0 | 39.7 | 25 | 0.67 | 1.05 | 3.18 | 9.61 | 15.1 |
| 26 | 4.13 | 5.87 | 13.8 | 32.4 | 46.0 | 26 | 0.83 | 1.31 | 3.96 | 12.0 | 18.8 |
| 27 | 4.79 | 6.79 | 16.0 | 37.5 | 53.3 | 27 | 1.04 | 1.64 | 4.94 | 14.9 | 23.5 |
| 28 | 5.54 | 7.87 | 18.5 | 43.5 | 61.7 | 28 | 1.30 | 2.04 | 6.15 | 18.6 | 29.2 |
| 29 | 6.42 | 9.11 | 21.4 | 50.4 | 71.5 | 29 | 1.61 | 2.54 | 7.67 | 23.2 | 36.4 |
| 30 | 7.43 | 10.6 | 24.8 | 58.3 | 82.8 | 30 | 2.01 | 3.16 | 9.56 | 28.9 | 45.4 |
| 31 | 8.61 | 12.2 | 28.7 | 67.5 | 95.8 | 31 | 2.51 | 3.94 | 11.9 | 36.0 | 56.6 |
| 32 | 9.97 | 14.1 | 33.3 | 78.2 | 111.0 | 32 | 3.12 | 4.91 | 14.8 | 44.0 | 70.5 |
| 33 | 11.5 | 16.4 | 38.5 | 90.5 | 129.0 | 33 | 3.89 | 6.12 | 18.5 | 55.8 | 87.8 |
| 34 | 13.4 | 19.0 | 44.6 | 105.0 | 149.0 | 34 | 4.85 | 7.63 | 23.0 | 69.6 | 108.0 |
| 35 | 15.5 | 22.0 | 51.6 | 121.0 | | 35 | 6.04 | 9.51 | 28.7 | 86.7 | 136.0 |
| 36 | 17.9 | 25.4 | 59.8 | 141.0 | | 36 | 7.53 | 11.8 | 35.8 | 108.0 | |
| 37 | 20.8 | 29.5 | 69.3 | | | 37 | 9.38 | 14.8 | 44.6 | 135.0 | |
| 38 | 24.0 | 34.1 | 80.2 | | | 38 | 11.7 | 18.4 | 55.5 | | |
| 39 | 27.8 | 39.5 | 92.9 | | | 39 | 14.6 | 22.9 | 69.2 | | |
| 40 | 32.2 | 45.7 | 108.0 | | | 40 | 18.2 | 28.6 | 86.2 | | |

In particular preferred embodiments of the invention the depression is treatment-refractory or treatment-resistant depressive disorder or a depression requiring rapid mood improvement (e.g. due to suicidal behavior), and in such cases the treatment further comprises a 5 step of administering to the subject a therapeutically effective amount of the compound or composition together with at least one, preferably one or two, additional antidepressant.

In some embodiments, the treatment (or patient selection) is entirely independent of the observed leptin levels of the subject. Further preferred is a treatment of a patient having physiological leptin levels; alternatively, a patient having increased leptin levels may be subject of the invention. The reference leptin level depends on age and sex of the individual and may vary. A clinician is however familiar with diagnosing a physiological (or normal/healthy) leptin level in an individual patient. The depressive disorder or depressive episode may thus occur preferably in a patient with a low, normal or high serum leptin level who does not have a primary or comorbid diagnosis of acute anorexia nervosa.

Preferred according to the invention is that the at least one additional antidepressant is independently selected from the group consisting of imipramine, amitriptyline, desipramine, nortriptyline, doxepin, protriptyline, trimipramine, maprotiline, amoxapine, trazodone, bupropion, chlomipramine, fluoxetine, dulloxetine, escitalopram, citalopram, sertraline, paroxetine, fluvoxamine, nefazadone, venlafaxine, milnacipran, reboxetine, lithium, mirtazapine, phenelzine, tranylcypromine, moclobemide, Kava-Kava, St. John's Wart, S-adenosylmethionine, thyrotropin releasing hormone, neurokinin receptor antagonists, triiodothyronine, and esketamine. Preferably, the at least one additional antidepressant is independently selected from the group consisting of mono-amine oxidase inhibitors, tricyclics, serotonin reuptake inhibitors, serotonin noradrenergic reuptake inhibitors; noradrenergic and specific serotonergic agents and atypical antidepressants. More preferably, the at least one additional antidepressant is independently selected from the group consisting of phenelzine, tranylcypromine, moclobemide, imipramine, amitriptyline, desipramine, nortriptyline, doxepin, protriptyline, trimipramine, chlomipramine, amoxapine, fluoxetine, Sertraline, paroxetine, citalopram, fluvoxamine, Venlafaxine, milnacipran, mirtazapine and bupropion.

Additionally or alternatively, the at least one antidepressant may further comprise an atypical antidepressant. The atypical antidepressant is preferably selected from the group consisting of aripiprazole, quetiapine, olanzapine, risperidone and paliperidone, preferably selected from the group consisting of aripiprazole, quetiapine and olanzapine.

In particular the invention also pertains, in an additional aspect, to a method for treating depression in a subject, the method comprising a use as recited in any one of the preceding aspects, in particular with respect to the subject to be treated, the administered compound or composition, or depression to be treated in the subject.

The term “administration” can refer to introducing a substance, such as leptin, fragments thereof, and/or leptin receptor agonists, or a composition comprising leptin, into a subject. Any route of administration may be utilized. Examples of routes of administration include parenteral, e.g., intravenous, intradermal, subcutaneous, oral, transdermal (topical), transmucosal, and rectal administration. In certain preferred embodiments the leptin, or the leptin- and/or leptin receptor agonist, or the leptin analog or derivative thereof, is administered once daily, or is administered in two or more dose administrations over one day, wherein the sum of doses administered does not exceed the daily dosage of metreleptin.

In embodiments, the leptin dose can be adjusted. For example, the leptin dose can be adjusted up or adjusted down. For example, the leptin dose can be adjusted up for a period of time, and then down for a period of time. In a clinical setting, for example, leptin can be administered at a dose of about 0.04 mg/kg/day for female patients and 0.02 mg/kg/day for male patients for the first four weeks and doubled after for an additional 16 weeks.

In further embodiments of the invention the administration of the leptin, or the leptin- and/or leptin receptor agonist, or the leptin analog or derivative thereof, is an administration of metreleptin or PEG-leptin (pegylated leptin) by subcutaneous injection.

The administration of a therapeutically effective amount of leptin, or the leptin agonist, analog or derivative thereof, to the subject so ameliorates in the subject one or more measures of a depressive disorder in the subject.

A “subject” or “patient” in context of the invention is preferably a mammal such as a primate. Preferred subjects are human subjects and include a female (or male) human.

Preferred subject indicated for a treatment of the invention is a human and wherein the subject does not suffer from or is not diagnosed with acute anorexia nervosa (AN), and preferably not AN, and preferably such a patient who does not suffer from or is not diagnosed with AN is not a patient with a pathological low body mass index (BMI), such as of less than about 20 kg/m², 19.0 kg/m², preferably of less than 18.0 kg/m², 17.5 kg/m², 16.5 kg/m², or less than 15 kg/m², 14 kg/m², 13 kg/m², or less than 12 kg/m², 11 kg/m² or 10 kg/m².

In another embodiments of the invention the subject indicated for a treatment according to the invention is a human patient and is distinguished by having a serum leptin concentration of about 4 ng/ml or more, preferably of about 5 ng/ml or more, most preferably of 5.5 ng/ml or more.

Human subjects indicated for the treatments of the invention are usually subjects suffering from mild, moderate or major depression, preferably who do not suffer from AN or are not diagnosed with AN.

The invention further pertains to the following itemized embodiments:

Item 1: A compound or composition for use in the treatment of a mood disorder in a human subject, wherein the compound or composition comprises a leptin, or a leptin- and/or leptin receptor agonist, or a leptin analog or derivative thereof; or wherein the compound or composition is for use in the treatment of a depressive episode.

Item 2: The compound or composition for use of item 1, wherein the mood disorder is selected from (1) a depressive disorder or depressive episode, or (2) from a bipolar disorder or bipolar related disorder, wherein preferably (2) is a bipolar disorder or bipolar related disorder with current depressive episode.

Item 3: The compound or composition for use of item 1 or 2, wherein the mood disorder is diagnosed as a primary disease in the human subject or is diagnosed as a secondary disease (comorbidity) in the human subject to be treated.

Item 4: The compound or composition for use of anyone of items 1 to 3, wherein the mood disorder is a depressive disorder or depressive episode which is otherwise treatment-refractory or treatment-resistant.

Item 5: The compound or composition for use of anyone of items 1 to 4, wherein the leptin, or the leptin- and/or leptin receptor agonist, or the leptin analog or derivative thereof, is administered at: from about 1 microgram per day, from about 5 micrograms per day, about 10 micrograms per day, from about 50 micrograms per day, or from about 100 micrograms per day; to about 100 micrograms per day, to about 500 micrograms per day, to about 1 milligram per day, to about 5 milligrams per day, to about 50 milligrams per day, or to about 100 milligrams per day; or from about 0.01 milligram per kilogram to about 0.3 milligram, or from about 0.01 milligram per kilogram to about 20 milligrams per kilogram.

Item 6: The compound or composition for use of anyone of items 1 to 5, wherein the leptin analog is metreleptin.

Item 7: The compound or composition for use of item 6, wherein metreleptin is administered at: from about 1 milligram per day, from about 5 milligrams per day, to about a maximum of 25 milligrams per day; or from about 0.06 milligram per kilogram per day to about 2.5 milligrams per kilogram per day; and/or wherein the metreleptin is administered at 0.06 mg/kg to 10 mg per day, preferably wherein metreleptin is administered at about 3 to 10 mg per day.

Item 8: The compound or composition for use of anyone of items 1 to 7, wherein the leptin, or the leptin- and/or leptin receptor agonist, or the leptin analog or derivative thereof, is administered once daily, or is administered in two or more dose administrations over one day.

Item 9: The compound or composition for use of any one of items 1 to 8, wherein the administration of the leptin, or the leptin- and/or leptin receptor agonist, or the leptin analog or derivative thereof, is an administration of metreleptin or PEG-leptin (pegylated leptin) by subcutaneous injection.

Item 10: The compound or composition for use of any one of items 1 to 9, wherein the human subject is a boy or girl, or a human adult man or -adult woman.

Item 11: The compound or composition for use of anyone of the preceding items, wherein the depression is treatment-refractory or treatment-resistant depression or a depression requiring rapid mood improvement (e.g. due to suicidal behavior), the treatment comprising a step of administering to the human subject a therapeutically effective amount of the compound or composition together with at least one, preferably one or two, additional antidepressant.

Item 12: The compound or composition for use of item 11, wherein the at least one additional antidepressant is independently selected from the group consisting of imipramine, amitriptyline, desipramine, nortriptyline, doxepin, protriptyline, trimipramine, maprotiline, amoxapine, trazodone, bupropion, chlomipramine, fluoxetine, dulloxetine, escitalopram, citalopram, sertraline, paroxetine, fluvoxamine, nefazadone, venlafaxine, milnacipran, reboxetine, lithium, mirtazapine, phenelzine, tranylcypromine, moclobemide, Kava-Kava, St. John's Wart, S-adenosylmethionine, thyrotropin releasing hormone, neurokinin receptor antagonists, triiodothyronine, and esketamine.

Item 13: The compound or composition for use of item 11 or 12, wherein the at least one additional antidepressant is independently selected from the group consisting of mono-amine oxidase inhibitors, tricyclics, serotonin reuptake inhibitors, serotonin noradrenergic reuptake inhibitors; noradrenergic and specific serotonergic agents and atypical antidepressants.

Item 14: The compound or composition for use any one of items 11 to 13, wherein the at least one additional antidepressant is independently selected from the group consisting of phenelzine, tranylcypromine, moclobemide, imipramine, amitriptyline, desipramine, nortriptyline, doxepin, protriptyline, trimipramine, chlomipramine, amoxapine, fluoxetine, Sertraline, paroxetine, citalopram, fluvoxamine, Venlafaxine, milnacipran, mirtazapine and bupropion.

Item 15: The compound or composition for use of anyone of items 11 to 14, wherein the at least one antidepressant further comprises an atypical antidepressant.

Item 16: The compound or composition for use of item 15, wherein the atypical antidepressant is selected from the group consisting of aripiprazole, quetiapine, olanzapine, risperidone and paliperidone, preferably selected from the group consisting of aripiprazole, quetiapine and olanzapine.

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, 10%, and for example 5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

### BRIEF DESCRIPTION OF THE FIGURES AND SEQUENCES

The figures show:

FIG. 1: shows BDI scores for patients with LD from T1 to T4 (n=10).

FIG. 2: shows Boxplots to BDI scores for patients with LD from the time before start of treatment with metreleptin and at the following three time points (n=10). The significant Bonferroni-corrected P-values based on the conditional post-hoc analyses (Friedman's test) are indicated.

FIG. 3: shows Leptin levels (log-transformed) and BDI scores at T1 for patients (n=10) with LD.

FIG. 4: shows means of depressive cognitions and emotions assessed twice daily with visual analogue scales (range 1-10) prior to, during and after the 9-day dosing period.

FIG. 5: shows means of eating disorder associated cognitions and emotions assessed twice daily with visual analogue scales (range 1-10) prior to, during and after the 9-day dosing period.

FIG. 6: shows means of physiological parameters assessed twice daily with visual analogue scales (range 1-10) prior, during and after the 24-day dosing period.

FIG. 7: shows the effect of metreleptin treatment on the depression of eleven patients (A, B, C, F, R, Y, L, PF, FU, T and M). (A) BDI-II scores before and after dosing of the patients. (B) Clinician-rated HAMD-17 or CDRS-R raw scores are provided. In case of HAMD-17 scores, a HAMD score of >=24 indicates a severe depression, a HAMD score of 17-23 indicates a moderate depression, a HAMD score of 8-16 indicates a mild depression and a HAMD score of 0-7 indicates no depression. In case of the CDRS-R data, a score above the horizontal line is indicative of depression.

The sequences show

Metreleptin amino acid sequence
SEQ ID NO: 1
MVPIQKVQDD TKTLIKTIVT RINDISHTQS VSSKQKVTGL
DFIPGLHPIL TLSKMDQTLA VYQQILTSMP SRNVIQISND
LENLRDLLHV LAFSKSCHLP WASGLETLDS LGGVLEASGY
STEVVALSRL QGSLQDMLWQ LDLSPGC

### EXAMPLES

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

The results are shown in Milos et al. 2020: “Short-term metreleptin treatment of patients with anorexia nervosa: rapid on-set of beneficial cognitive, emotional, and behavioral effects” Translational Psychiatry volume 10, Article number: 303 (2020), as well as in Antel, J., et al., Eur Child Adolesc Psychiatry (2021), both incorporated herein in their entirety.

The examples show:

### Example 1: Treatment of Depression in Lipodystrophy Patients

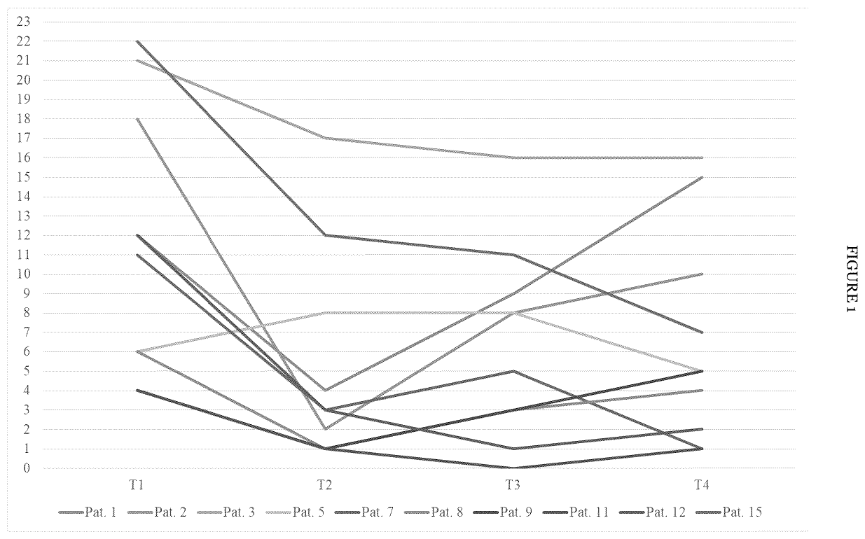

Descriptive data are presented in Table 1. The sample comprised ten patients with PLD nine women and one man. The mean age was 42.1 years. BDI scores from the time before start of treatment with metreleptin and at the following three time points are shown in the Table 2 and in FIG. 1. Of ten patients, four had mild depression (patients no. 1, 2, 7 and 12) and two had moderate depression (patients no. 3 and 15) in T1.

TABLE 1
Descriptive data for study group with patients with PLD
from T1 (prior to initiation of metreleptin treatment)
and at the following three time points (T2-T4; n = 10).

| | N (%) |
|---|---|
| Women/men | 9/1 (90%; 10%) |
| | Mean (SD) range |
| Age T1 (years) | 42.05 (11.20) 23.04-55.29 |
| Days between T1 and T2 | 7.70 (2.75) 6-15 |
| Days between T1 and T3 | 31.10 (5.38) 27-42 |
| Days between T1 and T4 | 91.10 (6.27) 83-99 |
| BMI [kg/m²] at T1 | 29.22 (2.86) 24.96-33.91 |
| BMI [kg/m²] at T2 | 28.22 (2.82) 24.96-33.34 |
| BMI [kg/m²] at T3 | 28.22 (2.89) 24.96-33.83 |
| BMI [kg/m²] at T4 | 29.22 (2.88) 24.96-33.87 |

TABLE 2
Beck Depression Inventory (BDI) scores for
patients with PLD from T1 to T4 (n = 10).

| | BDI | | | |
|---|---|---|---|---|
| | (Mean (SD) | 25. | 50. | 75. |
| Time | range) | percentile | percentile | percentile |
| T1 | 11.60 (6.80) 4-22 | 5.50 | 11.50 | 18.75 |
| T2 | 5.20 (2.45) 1-17 | 1.00 | 3.00 | 9.00 |
| T3 | 6.40 (4.94) 0-16 | 2.50 | 6.50 | 9.50 |
| T4 | 6.60 (6.44) 1-16 | 1.75 | 5.00 | 11.25 |

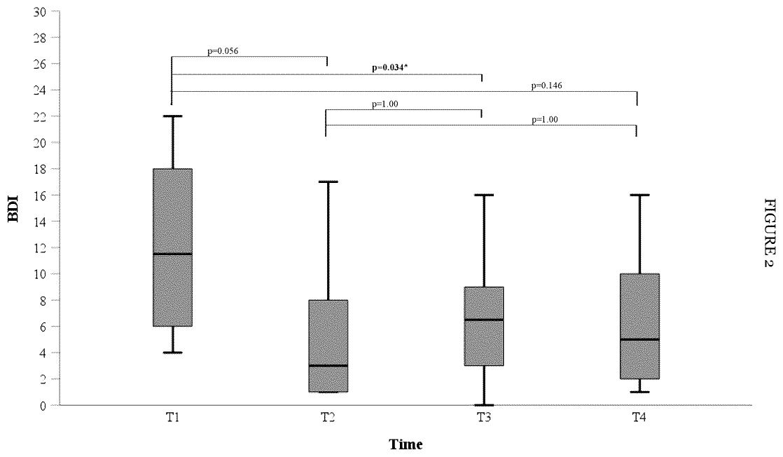

The nonparametric Friedman test revealed significant differences in BDI scores between the four time points (λ2(df=3)=10.268; p=0.012). Mean ranks were: T1=3.60, T2=2.10, T3=2.00 and T4=2.39. Post-hoc analyses revealed that only the difference between T1 and T3 was significant (Bonferroni corrected p=0.034, effect size r=0.88) (FIG. 2).

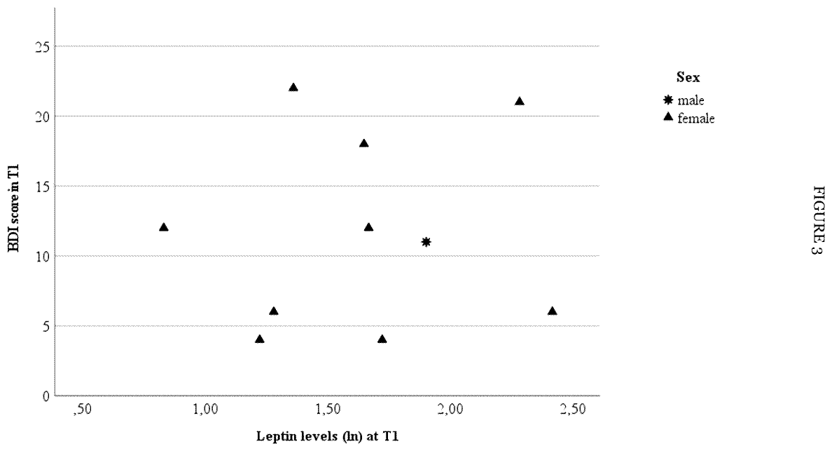

Spearman's correlation between leptin levels (In-transformed) and BDI at T1 was ρ=0.04 (p=0.920) (FIG. 3).

The inventors conducted sensitivity analyses with BDI, where questions R and S were excluded. Again, the Friedman test revealed significant differences in the revised BDI scores between time points T1 to T4 (λ2 (3)=12.41, p=0.003). Post-hoc analyses revealed significant differences in revised BDI scores between time points T1 vs. T2 (Bonferroni corrected p=0.002, r=0.99) and T1 vs. T3 (p=0.007, r=0.79).

This study for the first time revealed an antidepressant effect of metreleptin in patients with PLD, thus substantiating the hypothesis of an antidepressant effect in patients of hypoleptinemia irrespective of its cause. BDI total scores were significantly lower after the initiation of metreleptin treatment; according to the present data this effect persists up to T4 (approximately three months after initiation of dosing). This improvement in BDI scores is most pronounced and statistically significant when comparing the periods prior to and after commencement of dosing (the comparison between T1 and T3 was significant upon Bonferroni correction). When considering only the revised BDI scores (excluding the questions regarding appetite and weight loss), the ANOVA was again significant. Metreleptin thus causes a rapid drop in depression scores, indicative of the antidepressant action of metreleptin in patients with GLD.

### Example 2: Case Report about a Treatment of Major Depression Using Metreleptin

The currently 17-year-old female patient J was initially referred at age 16 with a severe episode of recurrent MDD with psychotic features and comorbid social anxiety disorder. Pronounced suicidal ideation in addition to recurrent deliberate self-harm made inpatient treatment necessary. J had not been able to attend school and had spent most of her time at home in her room/bed. She reported episodes of body dissatisfaction, dieting, and exercising but did not meet diagnostic criteria for an eating disorder. She had previously been diagnosed with bipolar disorder and had been taking valproic acid. However, we were unable to verify the lifetime occurrence of mania or hypomania—accordingly, medication with valproic acid was terminated; the patient was started on citalopram (up to 20 mg/d). Upon initial admission, J had a BMI of 25.82 kg/m² (BMI age percentile 93); she perceived herself as overweight and described recurrent episodes of binge eating and occasional vomiting. Repeated intentional weight loss and subsequent weight regain resulted in a BMI range of 20.44 (BMI age percentile 40) to 25.82 kg/m² prior to referral.

Treatment consisted of individual and family-based psychotherapy and medication.

During the three months long inpatient treatment episode the patient was switched to quetiapine (100 mg/d) in light of no beneficial effect of citalopram. After a moderate improvement of mood and anxiety, the patient was discharged, only to be readmitted approximately four weeks later. In addition to recurrence of a severe episode of MDD, she reported a marked increase in binge eating and self-induced vomiting and was thus diagnosed with beginning bulimia nervosa (BN). To improve both symptoms of depression and BN the patient was additionally started on fluoxetine 10 mg/day. Inpatient treatment was terminated after seven days on the patient's request.

After discharge, she was also treated with psychotherapy weekly; she nevertheless described a worsening of mood, daily functioning and quality of life. Accordingly, fluoxetine and quetiapine were gradually increased to maximum doses of 30 mg/day and 200 mg/day. Three months later, the patient attempted suicide by ingestion of an overdose of her medication and was treated at a different hospital. Medication was stopped to avoid renewed suicidal ingestion of her medication.

The patient continued to describe severe symptoms of depression and school attendance was initially irregular, until she completely ceased to attend. Binge eating episodes and self-induced vomiting occurred three or four times a week; the patient lost approximately seven kilograms. At that time the patient described irregular menses and temporary cessation of menses for less than three months. Repeatedly determined serum leptin levels showed substantial fluctuation between ages 16/11 and 17/8, which likely reflect changes in both energy intake and fat mass.

Metreleptin dosages of 6 to 9 mg/d (6 mg:d1-d5; 9 mg:d6-d9) were applied subcutaneously once a day at 9:00 am; dosages did not exceed the maximal recommended metreleptin dosage of 10 mg/d for lipodystrophy in females 40 kg (https://www.bfarm.de/SharedDocs/Downloads/DE/Arzneimittel/Pharmakovigilanz/Risikoinf ormationen/EducationMaterial/Anlagen/metreleptin-myalepta-aerztei.pdf?_blob=publicationFile&v=3)

The inventors used visual analogue scales (VAS; daily means of morning and evening scores) to assess psychopathology on a daily basis.

The patient also self-rated depressive symptoms and eating disorder specific symptomatology using Beck Depression Inventory-II (Hautzinger et al. 2006) and Eating Disorder Inventory-2 (Thiel et al. 1997), respectively, two days prior to the intervention (d-2), six days into the dosing period (d6), one week (d+7) and two months after the last application. We obtained clinician-ratings of depression with the Childhood Depression Rating Scale-Revised three days prior (d-3) and on day eight (d8) of the dosing period.

The patient's mother filled in the Child Behavior Checklist (Achenbach 1999) to assess behavioural and emotional problems and competencies at two timepoints (−d2, d9). We repeatedly measured fasting serum leptin and BDNF levels. Glucose levels were monitored continuously from day 1 to day 9 (Freestyle Libre 2®).

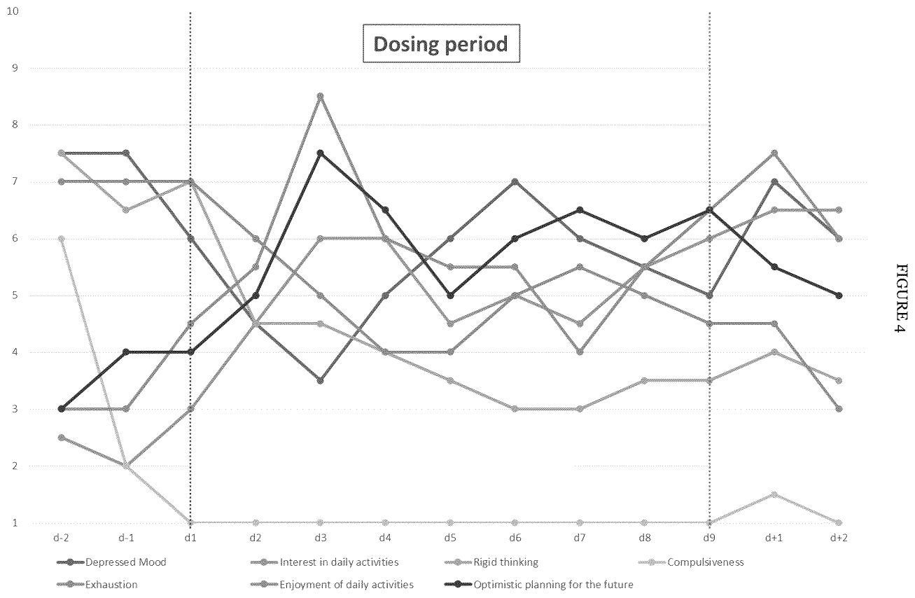

Symptoms of depression: Upon treatment initiation, mood and enjoyment of daily activities improved within one day. The patient reported less rigid thinking and compulsiveness and started to make plans for her future (FIG. 4). The patient also described “feeling less exhausted”. The patient herself, family members and treatment staff described a marked increase in willingness to engage in conversation and greater openness. However, especially the latter seemed to lead to too many social interactions and therewith an increase in social anxiety and irritation, which may in turn have led to a renewed increase in depressive symptoms as rated by VAS (FIG. 4, depressed mood). The patient requested to discontinue inpatient treatment after nine days; for safety reasons we decided against continuation of metreleptin dosing on an outpatient basis.

Both clinician (CDRS-R) and self-ratings (BDI-II) of depression decreased rapidly and substantially after onset of dosing and remained lower over time (Table 3). The patient nevertheless persistently uttered skepticism regarding the effectiveness of treatment, this being in accordance with previous psychopharmacological and psychotherapeutic treatment episodes.

TABLE 3
Clinician (Children's Depression Rating Scale - Revised (CDRS-R)), parent-rated (Child
Behaviour Checklist (CBCL) subscale Anxious/Depressed, Withdrawn/Depressed and self-rated
(Beck Depression Inventory-II (BDI-II); Eating Disorder Inventory (EDI-2)) scores.

| Day | d −3 | d −2 | d 6 | d 8 | d 9 | d +7 | d +81 |
|---|---|---|---|---|---|---|---|
| CDRS-R; Raw score/percentile | 86/100 | | | 62/100 | | | |
| rank | | | | | | | |
| BDI-II; total score | | 44 | 27 | | | 36 | 33 |
| CBCL Anxious/Depressed; | | 81 | | | 78 | | |
| T-score | | | | | | | |
| CBCL Withdrawn/Depressed; | | 83 | | | 76 | | |
| T-Score | | | | | | | |
| EDI-2 raw score/percentile rank | 359/99 | | 346/99 | | | 350/99 | 341/99 |

d = day in relationship to the nine day long dosing period;
−/+ = prior/after dosing

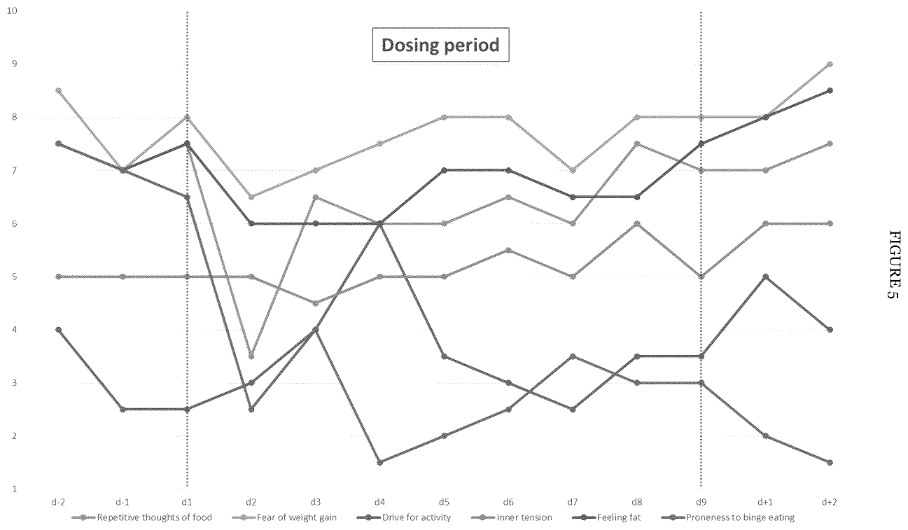

Eating disorder cognitions and emotions: The patient stopped self-induced vomiting during the nine days long inpatient treatment episode, during which she lost one kilogram due to restrictive eating. The patient's mother described a less compulsive eating behaviour with episodes of “normal”, carefree eating during visits to the hospital and during the single weekend stay at home. Corresponding with these observations, binge-eating decreased during and shortly after the dosing period (FIG. 5). Drive for activity was moderate prior to treatment and remained practically unchanged. Dosing did not affect self-rated eating disorder cognitions and emotions (see VAS items ‘feeling fat’, ‘fear of weight gain’). Overall, total raw scores of the EDI-2 remained largely stable (Table 1). The raw score for the subscale ineffectiveness was the only subscale score that showed a difference of more than five (46 at d-3; 39 at d7).

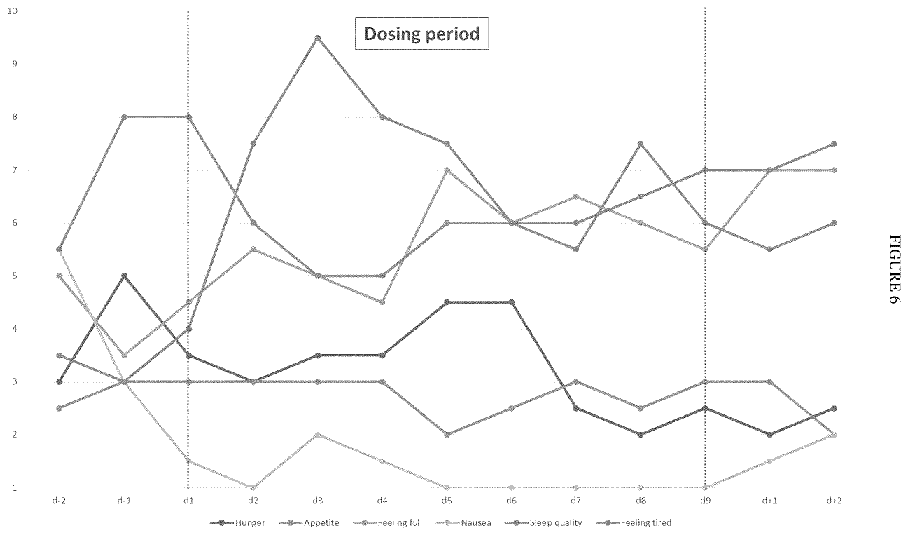

Physiologic effects: Appetite was continuously rated as low. Sleep quality improved substantially during the dosing period and the patient felt less exhausted (FIG. 6). Continuous glucose monitoring detected no episodes of hypoglycaemia (data not shown).

Serum leptin and BDNF levels: Circulating leptin levels were low prior to treatment, reached high levels two to six hours after metreleptin application and returned to low levels in the morning prior to the next dosing. BDNF levels at d-2 and d-1 proved divergent, with the initial value being low. At the end of the dosing period, the BDNF level was in the low normal range.

Follow-up: Ten weeks after discharge the patient had gained weight again and described less binge eating episodes and self-induced vomiting; the EDI-2 raw score was slightly lower than at baseline; the percentile rank remained at 99 throughout the observation period. The score of the subscale ‘body dissatisfaction’ was reduced (percentile rank dropped from 95 to 75 at d+81; Table 3).

The patient reported increased interest in daily activities but otherwise unchanged symptoms of depression. However, she had resumed school attendance and had started meeting with friends again. The parent's mother described clearly discernible changes in both mood and interest in activity. Notably, self-ratings of depressive symptoms revealed lower values than prior to dosing (Table 3).

Serum leptin was within the normal range; the serum BDNF level reached a high-normal value (d+81).

This case report describes the first-in-human metreleptin treatment of MDD and BN. Beneficial effects of treatment were evident to parents and treatment staff and to a lesser degree to the 17-year-old patient herself, who as in previous treatment episodes and with other treatment modalities expressed her ambivalence in terms of therapeutic progress. Depressive symptoms improved within the short dosing period of nine days and remained lower after discontinuation of treatment. The BDI-II score dropped from 44 two days prior to the dosing period to 27 on day six and remained at a reduced value of 33 two months after the last application. According to the test manual (Hautzinger und Keller et al. 2006), a difference of eight points in BDI-II scores represents a significant individual change. The National Institute for Health and Care Excellence (NICE) suggests an even smaller difference of 3 BDI-II points as a clinically significant treatment effect for normal depression, and a difference of two points for treatment-resistant depression (National Collaborating Centre for Mental Health 2004). The antidepressant effects were clearly less pronounced than in metreleptin treated patients with AN and comorbid depression, some of whom experienced a euphoric mood after being treated for three to five days (Milos et al., 2020; Antel et al., 2021).

### Example 3: Further Case Report of a Female Depression Patient

A female patient from the Arabian Peninsula developed extreme obesity during infancy as a result of hyperphagia. Congenital leptin deficiency was diagnosed at age 38 by molecular genetic testing using next generation sequencing of the genes KSR2, LEP, LEPR, MC4R, MRAP2, NTRK2, PCSK1, POMC and SIM1 (Illumina); her niece with a similar phenotype had initially been diagnosed with this disorder. Genetic sequencing of the leptin gene revealed a novel biallelic homozygous rare variant. The annotation tools MutationTaster and PolyPhen2 classified this variant as probably damaging. Functional characterization has been performed in our laboratory (MW) and is unpublished.

Upon initiation of metreleptin treatment at the Division of Pediatric Endocrinology Diabetes at the University Medical Center in Ulm, body height, weight and BMI of the index patient were 167.4 cm, 128.8 kg and 45.9 kg/m². She had hypertension, hypothyroidism, pancreas lipomatosis, steatosis hepatis, lipoedema grade 3, Sjögren syndrome and spondylolisthesis; she had been medicated with L-thyroxin (125 μg/day), vitamin B12 (1000 μg/day), vitamin D3 (50.000 IU/week).

This is the first detailed report of the psychological changes experienced by a patient with CLD upon initiation of metreleptin treatment. An interview with the patient was conducted during dosing day 14; the accounts of the 39-year old female are vivid and readily convey why she has experienced these changes as “magic”. Content wise, the amazement as to the reduced preoccupation with food and the ensuing mental liberation were readily perceptible. Throughout the interview, she repeatedly spoke of the seemingly all-encompassing extent to which she had been engaged with her preoccupation with food. Whereas she viewed her improved mood as a consequence of this reduced preoccupation, other changes were deemed as not being directly related (see paragraph on self-organization). According to the patient's recollection the initial psychological effects of metreleptin were experienced within three days.

In conclusion, this case report substantiates profound psychological effects of treatment with human recombinant leptin; in contrast to patients with AN who develop hypoleptinemia as a result of loss of fat mass, patients with CLD have inborn leptin deficiency.

### Example 4: Treatment with Metreleptin Improves BDI-II, HAMD-17 and CDRS-R Scores

To evaluate the effect of metreleptin treatment on anorexia nervosa (AN) patients, the BDI-II, HAMD-17 and CDRS-R scores of 11 anorexia nervosa patients (A, B, C, F, R, Y, L, PF, FU, T and M) for overall 14 dosing periods were examined.

Details on the treatment of patients A-C are found in Milos et al, “Short-term metreleptin treatment of patients with anorexia nervosa: rapid on-set of beneficial cognitive, emotional, and behavioral effects” Translational Psychiatry volume 10, Article number: 303 (2020). Details on the treatment of patient F are found in Antel et al., Eur Child Adolesc Psychiatry (2021).

Details on the treatment of patient R are found in Gradl-Dietsch et al., “Rapid Emergence of Appetite and Hunger Resulting in Weight Gain and Improvement of Eating Disorder Symptomatology during and after Short-Term Off-Label Metreleptin Treatment of a Patient with Anorexia Nervosa” Obesity Facts volume 16, No. 1, pages 99-107 (2023). This publication is incorporated herein in its entirety.

The results shown for Patients L, Y, PF, FU, T and M are based on unpublished data. Patients T and M were treated more than once. Patient T exhibited a comorbidity with post-traumatic stress disorder and obsessive compulsive disorder. Patient M was 19 years old at the time of dosing with a body weight of 25 kg.

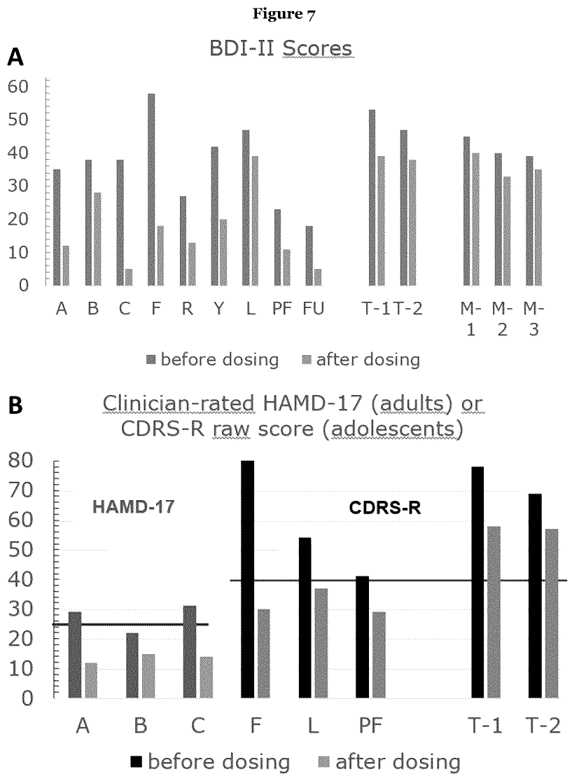

The BDI-II scores for the patients before and after treatment are shown in FIG. 7A. In this figure, a BDI-II score of 29-63 indicates a severe depression, a BDI-II score of 20-28 indicates a moderate depression, a BDI-II score of 14-19 indicates a minor depression, a BDI-II score of 9-13 indicates a minimal depression and a BDI-II score of 0-8 indicates that the patient suffers from no depression. The National Institute for Health and Care Excellence (NICE) suggests a difference of ≥3 BDI-II points is a clinically significant treatment effect for normal depression. Based on this rule, all patients exhibited a significant treatment effect after the respective dosing periods with metreleptin.

In the case that clinical ratings were available (A, B, C, F, L, PF and T), the effect of metreleptin treatment was further evaluated according to the CDRS-R (for adolescent patients) and HAMD-17 (for adult patients) scales. Details on the 17-item Hamilton Depression Rating Scale (HAMD-17) can be found in Zimmermann et al., “Severity classification on the Hamilton Depression Rating Scale” Journal of Affective Disorders, volume 150, issue 2, pages 384-388 (2013). In all of patients A, B and C, the HAMD-17 scores were reduced after treatment with metreleptin. In case of patients A and C, a severe depression was reduced to a mild depression. In FIG. 7(B), a CDRS-R score of ≥40 is indicative of depression, whereas a CDRS-R score of ≤28 is used to define remission (minimal or no symptoms). For all of the patients F, L, PF and T, the CDRS-R scores were reduced after metreleptin treatment. Furthermore, in the case of patients F, L and PF, the depression score was reduced below the threshold that is indicative of depression.

1. A method of treatment of a mood disorder in a human subject, the method comprising a step of administering a compound or composition to the human subject, wherein the compound or composition comprises a leptin, or a leptin- and/or leptin receptor agonist, or a leptin analog or derivative thereof; or wherein the compound or composition is for use in the treatment of a depressive episode.

2. The method of claim 1, wherein the mood disorder is selected from (1) a depressive disorder or depressive episode, or (2) from a bipolar disorder or bipolar related disorder, wherein preferably (2) is a bipolar disorder or bipolar related disorder with current depressive episode.

3. The method of claim 1, wherein the mood disorder is diagnosed as a primary disease in the human subject or is diagnosed as a secondary disease (comorbidity) in the human subject to be treated.

4. The method of claim 1, wherein the mood disorder is a depressive disorder or depressive episode which is otherwise treatment-refractory or treatment-resistant.

5. The method of claim 1, wherein the leptin, or the leptin- and/or leptin receptor agonist, or the leptin analog or derivative thereof, is administered at: from about 1 microgram per day, from about 5 micrograms per day, about 10 micrograms per day, from about 50 micrograms per day, or from about 100 micrograms per day; to about 100 micrograms per day, to about 500 micrograms per day, to about 1 milligram per day, to about 5 milligrams per day, to about 50 milligrams per day, or to about 100 milligrams per day; or from about 0.01 milligram per kilogram to about 0.3 milligram, or from about 0.01 milligram per kilogram to about 20 milligrams per kilogram.

6. The method of claim 1, wherein the leptin analog is metreleptin.

7. The method of claim 6, wherein metreleptin is administered at: from about 1 milligram per day, from about 5 milligrams per day, to about a maximum of 25 milligrams per day; or from about 0.06 milligram per kilogram per day to about 2.5 milligrams per kilogram per day; and/or wherein the metreleptin is administered at 0.06 mg/kg to 10 mg per day, preferably wherein metreleptin is administered at about 3 to 10 mg per day.

8. The method of claim 1, wherein the leptin, or the leptin- and/or leptin receptor agonist, or the leptin analog or derivative thereof, is administered once daily, or is administered in two or more dose administrations over one day.

9. The method of claim 1, wherein the administration of the leptin, or the leptin- and/or leptin receptor agonist, or the leptin analog or derivative thereof, is an administration of metreleptin or PEG-leptin (pegylated leptin) by subcutaneous injection.

10. The method of claim 1, wherein the human subject is a boy or girl, or a human adult man or adult woman.

11. The method of claim 1, wherein the depression is treatment-refractory or treatment-resistant depression or a depression requiring rapid mood improvement (e.g. due to suicidal behavior), the treatment comprising a step of administering to the human subject a therapeutically effective amount of the compound or composition together with at least one, preferably one or two, additional antidepressant.

12. The method of claim 11, wherein the at least one additional antidepressant is independently selected from the group consisting of imipramine, amitriptyline, desipramine, nortriptyline, doxepin, protriptyline, trimipramine, maprotiline, amoxapine, trazodone, bupropion, chlomipramine, fluoxetine, dulloxetine, escitalopram, citalopram, sertraline, paroxetine, fluvoxamine, nefazadone, venlafaxine, milnacipran, reboxetine, lithium, mirtazapine, phenelzine, tranylcypromine, moclobemide, Kava-Kava, St. John's Wart, S-adenosylmethionine, thyrotropin releasing hormone, neurokinin receptor antagonists, triiodothyronine, and esketamine.

13. The method of claim 11, wherein the at least one additional antidepressant is independently selected from the group consisting of mono-amine oxidase inhibitors, tricyclics, serotonin reuptake inhibitors, serotonin noradrenergic reuptake inhibitors; noradrenergic and specific serotonergic agents and atypical antidepressants.

14. The method of claim 11, wherein the at least one additional antidepressant is independently selected from the group consisting of phenelzine, tranylcypromine, moclobemide, imipramine, amitriptyline, desipramine, nortriptyline, doxepin, protriptyline, trimipramine, chlomipramine, amoxapine, fluoxetine, Sertraline, paroxetine, citalopram, fluvoxamine, Venlafaxine, milnacipran, mirtazapine and bupropion.

15. The method of claim 11, wherein the at least one antidepressant further comprises an atypical antidepressant.

16. The method of claim 15, wherein the atypical antidepressant is selected from the group consisting of aripiprazole, quetiapine, olanzapine, risperidone and paliperidone, preferably selected from the group consisting of aripiprazole, quetiapine and olanzapine.
